(19) United States
(12) Patent Application Publication
(10) Pub. No.: US 2022/0168542 A1
(43) Pub. Date: Jun. 2, 2022

(54) INTRAVASCULAR ULTRAVIOLET CATHETER AND METHODS FOR MAKING AND USING THE SAME

(71) Applicant: BOSTON SCIENTIFIC SCIMED, INC., Maple Grove, MN (US)

(72) Inventors: Dongming HOU, Plymouth, MN (US); Tim O'CONNOR, Co. Galway (IE); Aiden FLANAGAN, Co. Galway (IE); Bryan Allen CLARK, Forest Lake, MN (US); Esteban Martin SOLORZANO ZELEDON, Shoreview, MN (US)

(73) Assignee: BOSTON SCIENTIFIC SCIMED, INC., Maple Grove, MN (US)

(21) Appl. No.: 17/539,471

(22) Filed: Dec. 1, 2021

Related U.S. Application Data
(60) Provisional application No. 63/120,056, filed on Dec. 1, 2020.

(51) Int. Cl. A61M 25/00

(52) U.S. Cl. CPC A61M 25/0017; A61M 2025/0024; A61M 25/0026

(57) ABSTRACT

Medical devices and methods for making and using medical devices are disclosed. An example device may include an elongate shaft having a distal end region. An expandable framework may be disposed adjacent to the distal end region. An ultraviolet light-emitting member may be disposed within the expandable framework. A covering may be disposed along an outer surface of the expandable framework.

### CROSS REFERENCE TO RELATED APPLICATIONS

The present application claims the benefit of and priority to U.S. Prov. Pat. App. No. 63/120,056, filed Dec. 1, 2020 and titled INTRAVASCULAR ULTRAVIOLET CATHETER AND METHODS FOR MAKING AND USING THE SAME, the disclosure of which is incorporated herein by reference.

### TECHNICAL FIELD

The present disclosure pertains to medical devices, and methods for manufacturing medical devices. More particularly, the present disclosure pertains to intravascular ultraviolet catheters.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A medical device is disclosed. The medical device comprises: an elongate shaft having a distal end region; an expandable framework disposed adjacent to the distal end region; an ultraviolet light-emitting member disposed within the expandable framework; and a covering disposed along an outer surface of the expandable framework.

Alternatively or additionally to any of the embodiments above, an intermediate shaft is disposed within the elongate shaft and wherein the expandable framework is coupled to the intermediate shaft.

Alternatively or additionally to any of the embodiments above, the elongate shaft is slidable relative to the intermediate shaft.

Alternatively or additionally to any of the embodiments above, an inner shaft is disposed within the elongate shaft and wherein the ultraviolet light-emitting member is coupled to the inner shaft.

Alternatively or additionally to any of the embodiments above, the covering is non-transparent to ultraviolet light.

Alternatively or additionally to any of the embodiments above, the covering extends along a first portion of the expandable framework.

Alternatively or additionally to any of the embodiments above, the ultraviolet light-emitting member is disposed adjacent to the first portion.

Alternatively or additionally to any of the embodiments above, a second portion of the expandable framework is free of the covering.

A medical device is disclosed. The medical device comprises: an elongate shaft having a distal end region and a lumen formed therein; wherein a first opening is formed along the distal end region, the first opening being configured to allow blood to flow into the lumen; wherein a second opening is formed along the distal end region, the second opening being configured to allow blood to flow out from the lumen; wherein the first opening is disposed proximally of the second opening; and one or more ultraviolet light-emitting members disposed along the lumen.

Alternatively or additionally to any of the embodiments above, the lumen has one or more curves formed therein.

Alternatively or additionally to any of the embodiments above, the lumen includes a first portion wherein blood entering the lumen through the first opening flows in a proximal-to-distal direction and a second section where blood entering the lumen through the first opening flows in a distal-to-proximal direction.

Alternatively or additionally to any of the embodiments above, the lumen includes a portion that splits into a plurality of lumens.

Alternatively or additionally to any of the embodiments above, the one or more ultraviolet light-emitting members include one or more optical fibers.

Alternatively or additionally to any of the embodiments above, at least one of the one or more optical fibers has an undulating shape.

Alternatively or additionally to any of the embodiments above, at least one of the one or more optical fibers is translatable relative to the shaft.

Alternatively or additionally to any of the embodiments above, at least one of the one or more optical fibers is rotatable relative to the shaft.

Alternatively or additionally to any of the embodiments above, at least one of the one or more optical fibers has a beveled distal end region.

A medical device is disclosed. The medical device comprises: a sheath having a ultraviolet light-transparent region; a shaft member disposed within the sheath, the shaft member having a sidewall aperture; an optical fiber disposed within the shaft member; and wherein the optical fiber has a beveled distal end region.

Alternatively or additionally to any of the embodiments above, the shaft member is translatable within the sheath.

Alternatively or additionally to any of the embodiments above, the optical fiber is rotatable relative to the shaft member.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a side view of an example medical device.

FIG. 2 is a side view of an example medical device.

FIG. 3 is a side view of an example medical device.

FIG. 4 is a side view of an example medical device.

FIG. 5 is a side view of an example medical device.

FIG. 7 is a side view of an example medical device.

FIG. 7 is a side view of an example medical device.

FIG. 8 is a side view of an example medical device.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

The SARS-CoV-2 virus, the virus that causes COVID-19, has reached pandemic levels in 2020. SARS-Co-V-2 uses the host ACE2 to enter and infect the host cells, including type 2 pneumocytes, macrophages, endothelial cells, pericytes, and cardiac myocytes. This may lead to immune system over-reactively, which may result in system inflammation, a storm of cytokine activity, and/or multiorgan failure. Disclosed herein are devices and methods aimed at killing SARS-CoV-2 virus (e.g., directly and/or indirectly such as via ultraviolet-induced DNA damage) and/or modulating the hyper-inflammatory response of the patient.

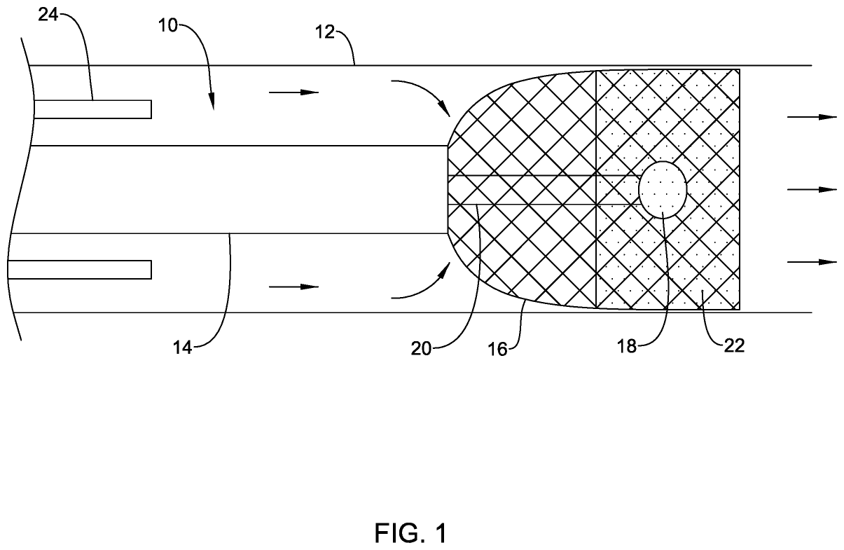

FIG. 1 is an example medical device 10 disposed within a blood vessel 12 (e.g., an artery or a vein). The medical device 10 may include a shaft 14 having an expandable framework 16 coupled thereto. An ultraviolet light-emitting member 18 may be coupled to an inner shaft 20. The ultraviolet light-emitting member 18 may be configured to be disposed adjacent to the expandable framework 16. A sleeve or covering 22 may be disposed along at least a portion of the expandable framework 16. In general, the medical device 10 is designed to be disposed in the blood vessel 12. The expandable framework 16 may be expanded and the ultraviolet light-emitting member 18 may be activated. Blood flowing through the blood vessel 12 and through the expandable framework 16 (e.g., as represented by arrows in FIG. 1) may be exposed to ultraviolet light emitted from the ultraviolet light-emitting member 18. In patients infected with SARS-Co-V-2, the ultraviolet light may help to reduce/eliminate SARS-Co-V-2 in the blood stream and/or help to reduce/attenuate SARS-Co-V-2.

The ultraviolet light-emitting member 18 may emit ultraviolet light using a suitable wavelength band such as UVA, UVB, and/or UVC. It may be desirable to expose about 100-1000 mL, or about 100-500 mL, or about 250-35 0mL of blood to ultraviolet light for about 1-120 seconds, or about 1-60 seconds, or about 5-30 seconds, or about 10 seconds.

In some instances, the shaft 14 may be an outer shaft of the medical device 10 and the inner shaft 20 may be movable relative to the shaft 14. In at least some of these instances, the expandable framework 16 may be coupled to the inner shaft 20. This may include coupling the proximal end of the expandable framework 16 to the inner shaft 20 at a position that is proximal of the distal end of the inner shaft 20 (e.g., and/or proximal of the ultraviolet light-emitting member 18). Movement of the shaft 14 relative to the inner shaft 20 can shift the medical device 10 between a first or delivery configuration (e.g., where the shaft 14 is advanced over the expandable framework 16, thereby collapsing and/or constraining the expandable framework 16) and a second or deployed configured (e.g., where the shaft is retracted relative to the inner shaft 20 so that the expandable framework 16 can expand within the blood vessel 12).

In other instances, the expandable framework 16 may be coupled to the distal end of the shaft 14. In such instances, the medical device may include an outer sheath 24, which may be slidable relative to the shaft 14 and can be used to shift the expandable framework 16 between the first and second configurations. When the expandable framework 16 is coupled to the shaft 14 (e.g., instead of the inner shaft 20), the ultraviolet light-emitting member 18 may be moved independently of the expandable framework 16.

As indicated above, a sleeve or covering 22 may be disposed along at least a portion of the expandable framework 16. In at least some instances, the covering 22 may substantially block ultraviolet light. Accordingly, the covering 22 may shield the wall of the blood vessel 12 from ultraviolet light during use of the medical device 10 (e.g., and/or during activation of the ultraviolet light-emitting member 18). In some instances, the covering 22 may be formed from polyurethane, polytetrafluoroethylene, polyethylene, combinations thereof, and/or other suitable materials including those disclosed herein.

It can be appreciated that the openings in the expandable framework 16 are designed to allow for blood to flow therethrough. In some instances, the expandable framework 16 may be designed to enhance turbulent flow of blow through therethrough. Turbulence may help to increase the volume of blood treated by the medical device 10. Other structures including the inner shaft 20, the shaft 14, and/or other components of the medical device 10 may also include features that are designed to help to enhance or promote turbulent flow. Such features may include coatings, texturing, and/or the like.

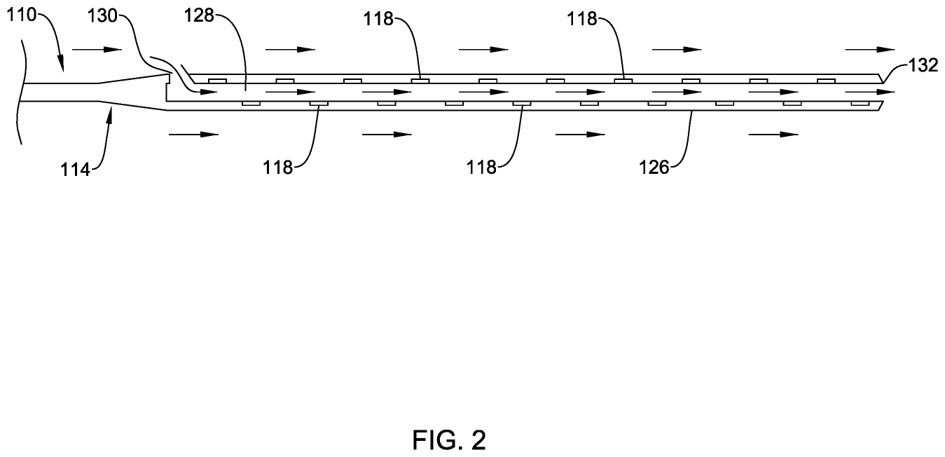

FIG. 2 illustrates another example medical device 110 that may be similar in form and function to other medical devices disclosed herein. The medical device 110 may include a shaft 114 having a distal end region 126. A passageway or lumen 128 may be defined within the shaft 114, for example within the distal end region 126. A first opening 130 may be formed in the shaft 114, for example along the distal end region 126. The first opening 130 may be configured to allow blood to flow into the lumen 128. A second opening 132 may be formed in the shaft 114, for example along the distal end region 126. The second opening 132 may be configured to allow blood to flow out from the lumen 128.

A plurality of ultraviolet light-emitting members 118 may be disposed adjacent to the lumen 128. In some instances, the plurality of ultraviolet light-emitting members 118 may be disposed along an interior wall or surface of the shaft 114, for example facing and/or adjacent to the lumen 128. As such blood can be exposed to ultraviolet light when flowing through the lumen 128.

The distal end region 126 may have a larger outer diameter than other portions of the shaft 114 and may have a length. These features may allow blood entering the lumen 128 to be exposed to ultraviolet light for a sufficient amount of time. Furthermore, in some instances, the openings 130, 132 along with the length of the shaft 114 (e.g., the length of the distal end region 126) may be designed so that the blood may enter the lumen 128 at a desired speed. For example, if the first opening 130 has a diameter of about 10 mm and blood enters the first opening 130 at a velocity of about 10 cm/sec, and if the diameter of the distal end region 126 is about 20 mm, blood entering the first opening 130 will slow when entering the lumen 128, for example to a velocity of about 2.5 cm/sec. If it is desired to expose blood to ultraviolet light for about 10 seconds, the distal end region 126 may have a length of about 25 cm or more. It can be appreciated that other arrangements of sizes can be utilized in order to expose blood to ultraviolet light for a desired amount of time.

In some instances, the distal end region 126 may include a cooling fluid lumen disposed adjacent to the lumen 128. The cooling fluid lumen may be in fluid communication with a coolant source. Infusing coolant into the cooling fluid lumen may help to cool blood passing through the lumen 128 (e.g., blood that may increase in temperature due to exposure to ultraviolet light).

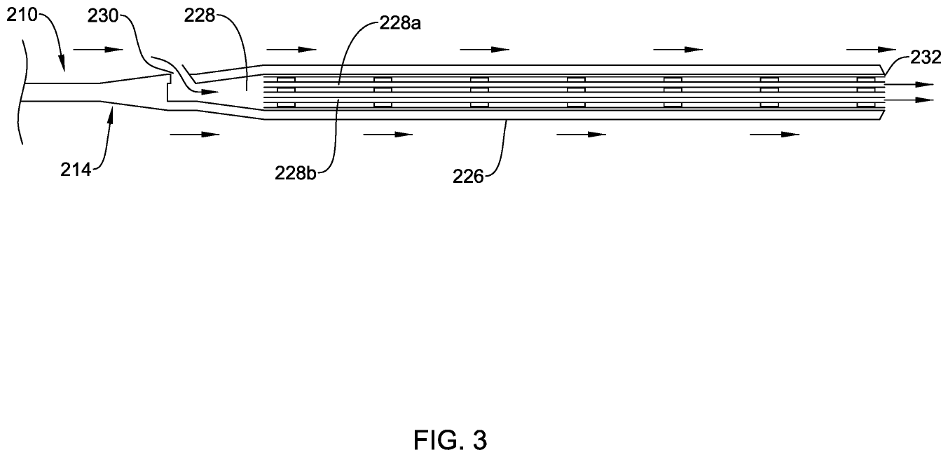

FIG. 3 illustrates another example medical device 210 that may be similar in form and function to other medical devices disclosed herein. The medical device 210 may include a shaft 214 having a distal end region 226. A passageway or lumen 228 may be defined within the shaft 214, for example within the distal end region 226. In some instances, the lumen 228 may segment or divide into a plurality of lumen sections, for example a first lumen section 228a and a second lumen section 228b.

A first opening 230 may be formed in the shaft 214, for example along the distal end region 226. The first opening 230 may be configured to allow blood to flow into the lumen 228. A second opening 232 may be formed in the shaft 214, for example along the distal end region 226. The second opening 232 may be configured to allow blood to flow out from the lumen 228.

A plurality of ultraviolet light-emitting members 218 may be disposed adjacent to the lumen 228. In some instances, the plurality of ultraviolet light-emitting members 218 may be disposed along an interior wall or surface of the shaft 214, for example facing and/or adjacent to the lumen sections 228a, 228b. As such blood can be exposed to ultraviolet light when flowing through the lumen 228.

In some instances, the distal end region 226 may include a cooling fluid lumen disposed adjacent to the lumen 228. The cooling fluid lumen may be in fluid communication with a coolant source. Infusing coolant into the cooling fluid lumen may help to cool blood passing through the lumen 228 (e.g., blood that may increase in temperature due to exposure to ultraviolet light).

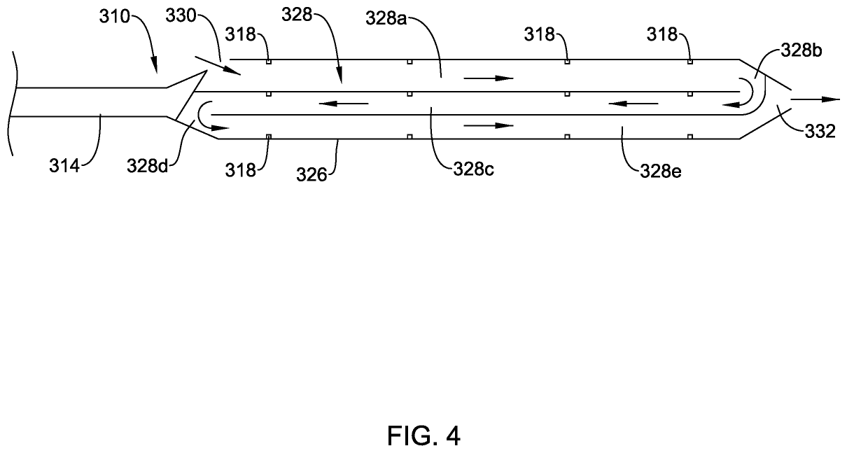

FIG. 4 illustrates another example medical device 310 that may be similar in form and function to other medical devices disclosed herein. The medical device 310 may include a shaft 314 having a distal end region 326.

A passageway or lumen 328 may be defined within the shaft 314, for example within the distal end region 326. In some instances, the lumen 328 may include a first section 328a, a first curve or curved region 328b, a second section 328c, a second curve or curved region 328d, and a third region 328e.

A first opening 330 may be formed in the shaft 314, for example along the distal end region 326. The first opening 330 may be configured to allow blood to flow into the lumen 328. A second opening 332 may be formed in the shaft 314, for example along the distal end region 326. The second opening 332 may be configured to allow blood to flow out from the lumen 328.

In some instances, the sections 328a, 328c, and 328e along with the curved regions 328b, 328d may allow blood to flow through the lumen 328 in different directions. For example, blood may flow in a proximal-to-distal direction along the first section 328a (e.g., and/or the third region 328e) and blood may flow in a distal-to-proximal direction along the second section 328c.

A plurality of ultraviolet light-emitting members 318 may be disposed adjacent to the lumen 328. In some instances, the plurality of ultraviolet light-emitting members 318 may be disposed along an interior wall or surface of the shaft 3214, for example facing and/or adjacent to the lumen 328. As such blood can be exposed to ultraviolet light when flowing through the lumen 328.

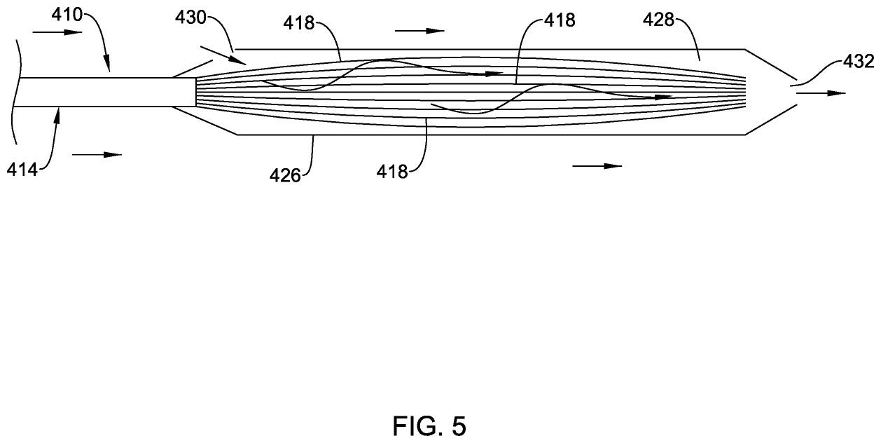

FIG. 5 illustrates another example medical device 410 that may be similar in form and function to other medical devices disclosed herein. The medical device 410 may include a shaft 414 having a distal end region 426. A first opening 430 may be formed in the shaft 414, for example along the distal end region 426. The first opening 430 may be configured to allow blood to flow into the lumen 428. A second opening 432 may be formed in the shaft 414, for example along the distal end region 426. The second opening 432 may be configured to allow blood to flow out from the lumen 428.

A plurality of ultraviolet light-emitting members 418 may be disposed adjacent to the lumen 428. In this example, the plurality of ultraviolet light-emitting members 418 may take the form of optical fibers. The optical fibers 418 may be arranged in a variety of different manners. For example, the optical fibers 418 may be follow a non-linear or arcuate pathway (e.g., between the first opening 430 and the second opening 432). Other arrangements are contemplated.

FIG. 6 illustrates another example medical device 510 that may be similar in form and function to other medical devices disclosed herein. The medical device 510 may include a shaft 514 having a distal end region 5426.

A first opening 530 may be formed in the shaft 514, for example along the distal end region 526. The first opening 530 may be configured to allow blood to flow into the lumen 528. A second opening 532 may be formed in the shaft 514, for example along the distal end region 526. The second opening 532 may be configured to allow blood to flow out from the lumen 528.

A plurality of ultraviolet light-emitting members 518 may be disposed adjacent to the lumen 528. In this example, the plurality of ultraviolet light-emitting members 518 may take the form of optical fibers. The optical fibers 518 may be arranged or otherwise include a plurality of undulations. For example, the optical fibers 518 may include undulations between the first opening 530 and the second opening 532. Other arrangements are contemplated. The undulations in the optical fibers 518 may help to increase turbulent flow of blood through the lumen 528.

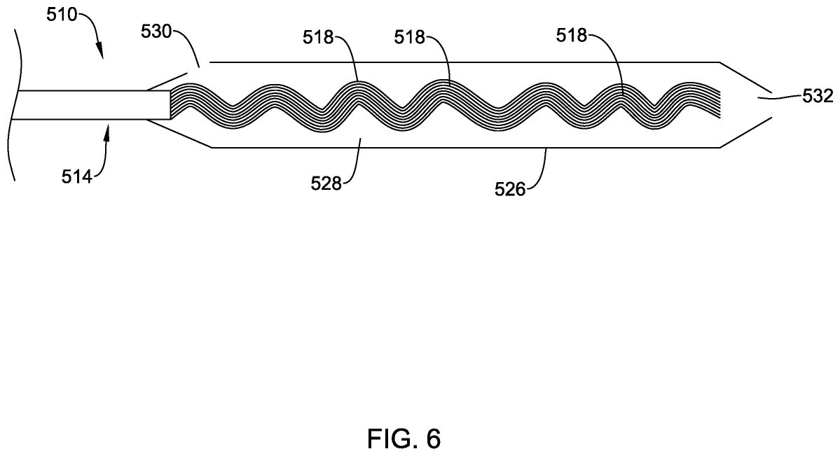

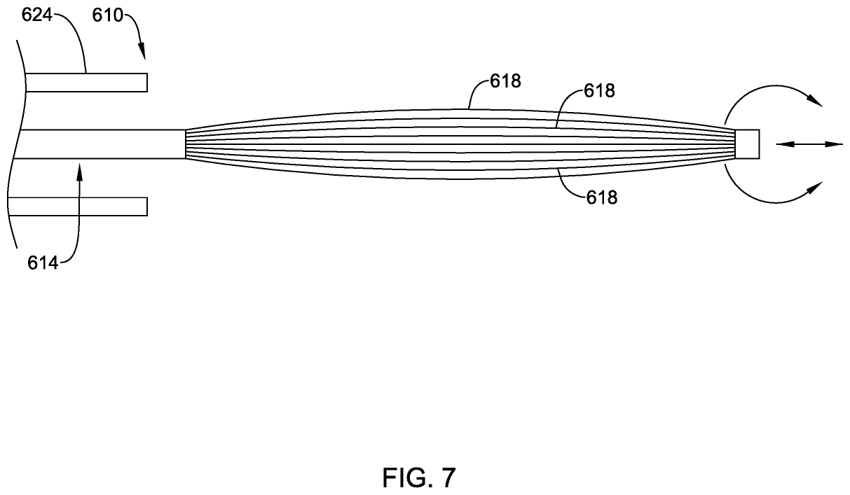

FIG. 7 illustrates another example medical device 610 that may be similar in form and function to other medical devices disclosed herein. The medical device 610 may include a shaft 614. A plurality of ultraviolet light-emitting members 618 may be coupled to the shaft 614. In this example, the plurality of ultraviolet light-emitting members 518 may take the form of optical fibers. The optical fibers 618 (e.g., and/or the shaft 614) may be translated and/or rotated. Rotation and/or translation of the optical fibers 618 may help to increase turbulence of blood.

An outer sheath 624 may be disposed along the shaft 614. The outer sheath 624 may be configured to shift between a first position or configuration (e.g., relative to the shaft 614) where the outer sheath 624 covers and/or contains the optical fibers 618 and a second position or configuration where the outer sheath 624 is disposed proximally of the optical fibers 618.

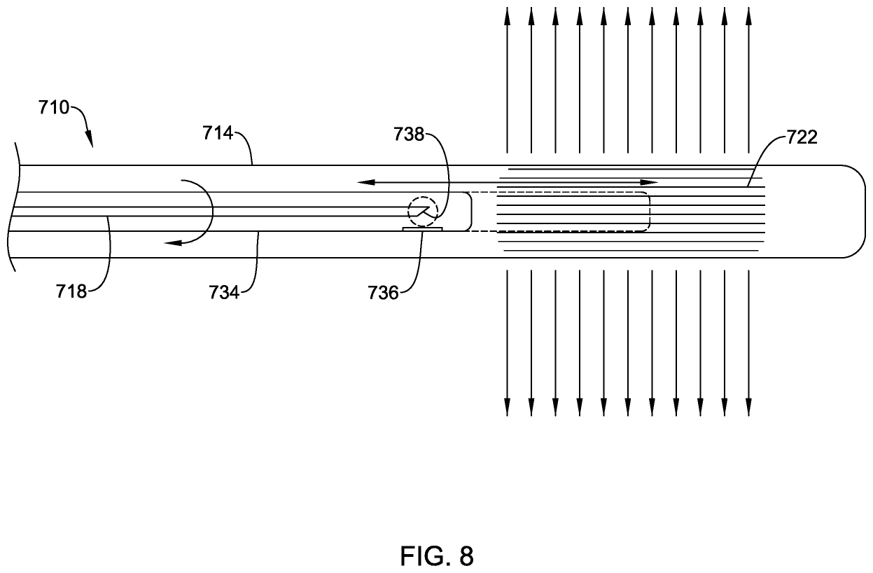

FIG. 8 illustrates another example medical device 710 that may be similar in form and function to other medical devices disclosed herein. The medical device 710 may include a shaft 714. An inner member 734 may be disposed within the shaft 714. An optical fiber 718 may be disposed within the inner member 734. The inner member 734 may include a sidewall opening or aperture 736. The optical fiber 718 may have a beveled end or end region 738. In at least some instances, the beveled end region 738 may include a mirror. The beveled shape and/or the mirror can help to direct light traveling along the optical fiber 718 at a desired angle toward the aperture 736.

The inner member 734 may be slidable within the shaft 714. In some instances, the inner member 734 may be advanced distally within the shaft 714 to a position adjacent to a window region 722 of the shaft 714. The window region 722 may help to control ultraviolet light transmission therethrough. For example, the window region 722 may be uniformly transparent to ultraviolet light. In other instances, the window region 722 may form a pattern that varies the transmission. For example, the window region 722 may have a horizontal slot pattern, a mesh pattern (e.g., a horizontal/vertical mesh pattern, a diamond-shaped mesh pattern, etc.), an attenuated or gradient pattern, a vertical slot pattern, combinations thereof, and/or the like.

The materials that can be used for the various components of the medical device 10 (and/or other medical devices disclosed herein) and the various tubular members disclosed herein may include those commonly associated with medical devices. The medical device 10 and/or other components thereof may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the medical device 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the medical device 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the medical device 10 to achieve the same result.

In some embodiments, a degree of Magnetic Resonance Imaging (MRI) compatibility is imparted into the medical device 10. For example, the medical device 10, or portions thereof, may be made of a material that does not substantially distort the image and create substantial artifacts (e.g., gaps in the image). Certain ferromagnetic materials, for example, may not be suitable because they may create artifacts in an MRI image. The medical device 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A medical device, comprising:
an elongate shaft having a distal end region;
an expandable framework disposed adjacent to the distal end region;
an ultraviolet light-emitting member disposed within the expandable framework; and
a covering disposed along an outer surface of the expandable framework.

2. The medical device of claim 1, wherein an intermediate shaft is disposed within the elongate shaft and wherein the expandable framework is coupled to the intermediate shaft.

3. The medical device of claim 2, wherein the elongate shaft is slidable relative to the intermediate shaft.

4. The medical device of claim 1, wherein an inner shaft is disposed within the elongate shaft and wherein the ultraviolet light-emitting member is coupled to the inner shaft.

5. The medical device of claim 1, wherein the covering is non-transparent to ultraviolet light.

6. The medical device of claim 1, wherein the covering extends along a first portion of the expandable framework.

7. The medical device of claim 6, wherein the ultraviolet light-emitting member is disposed adjacent to the first portion.

8. The medical device of claim 6, wherein a second portion of the expandable framework is free of the covering.

9. A medical device, comprising:
an elongate shaft having a distal end region and a lumen formed therein;
wherein a first opening is formed along the distal end region, the first opening being configured to allow blood to flow into the lumen;
wherein a second opening is formed along the distal end region, the second opening being configured to allow blood to flow out from the lumen;
wherein the first opening is disposed proximally of the second opening; and
one or more ultraviolet light-emitting members disposed along the lumen.

10. The medical device of claim 9, wherein the lumen has one or more curves formed therein.

11. The medical device of claim 9, wherein the lumen includes a first portion wherein blood entering the lumen through the first opening flows in a proximal-to-distal direction and a second section where blood entering the lumen through the first opening flows in a distal-to-proximal direction.

12. The medical device of claim 9, wherein the lumen includes a portion that splits into a plurality of lumens.

13. The medical device of claim 9, wherein the one or more ultraviolet light-emitting members include one or more optical fibers.

14. The medical device of claim 13, wherein at least one of the one or more optical fibers has an undulating shape.

15. The medical device of claim 13, wherein at least one of the one or more optical fibers is translatable relative to the shaft.

16. The medical device of claim 13, wherein at least one of the one or more optical fibers is rotatable relative to the shaft.

17. The medical device of claim 13, wherein at least one of the one or more optical fibers has a beveled distal end region.

18. A medical device, comprising:
a sheath having a ultraviolet light-transparent region;
a shaft member disposed within the sheath, the shaft member having a sidewall aperture;
an optical fiber disposed within the shaft member; and
wherein the optical fiber has a beveled distal end region.

19. The medical device of claim 18, wherein the shaft member is translatable within the sheath.

20. The medical device of claim 18, wherein the optical fiber is rotatable relative to the shaft member.
